(19) United States
(12) Patent Application Publication
(10) Pub. No.: US 2016/0242656 A1
(43) Pub. Date: Aug. 25, 2016

(54) Wrist Vital Monitoring Device

(71) Applicants: W. Charles Jackson, Pembroke Pines, FL (US); David L. Jackson, Minneapolis, MN (US)

(72) Inventors: W. Charles Jackson, Pembroke Pines, FL (US); David L. Jackson, Minneapolis, MN (US)

(21) Appl. No.: 15/097,283

(22) Filed: Apr. 12, 2016

Related U.S. Application Data
(60) Provisional application No. 62/115,708, filed on Feb. 13, 2015.
(60) Provisional application No. 62/172,192, filed on Jun. 7, 2015.

(51) Int. Cl. A61B 5/0205; A61B 5/1455; A61B 5/11; A61B 5/00

(52) U.S. Cl. CPC A61B 5/02055; A61B 5/6825; A61B 5/746; A61B 5/0015; A61B 5/14551; A61B 5/1112

(57) ABSTRACT

The present invention relates to a device worn about the wrist that monitors the user's vital signs, such as pulse, heart rate, blood pressure, temperature, and other vital signs. The device provides for the real time transfer of data relating to a user's vital signs to an external monitoring system. The external monitoring system may be staffed by certified medical personnel.

### CROSS-REFERENCE TO RELATED APPLICATIONS

This application claims the benefit of the priority to provisional application No. 62/115,708 entitled “Zatar” and 62/172,192 entitled “Zatar 3.0” filed with the United States Patent and Trademark Office on Feb. 13, 2015 and Jun. 7, 2015, respectively. The disclosure of these two applications is incorporated herein by reference as if fully set forth herein.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM

Not applicable.

### STATEMENT REGARDING PRIOR DISCLOSURES BY INVENTOR OR JOINT INVENTOR

Neither the inventor nor the joint inventor herein disclosed the invention herein prior to the 12 month period preceding the filing of their provisional applications, which are cross-referenced in paragraph b, above.

### BACKGROUND OF THE INVENTION

1. Field of the Invention

This application relates generally to portable monitoring devices that monitor the vital signs of a user. Disclosed is a portable measuring device which can be worn about the wrist and transmits through wireless communication data relating to the vital signs of the user. The following are some of the vital signs that can be measured: dynamic pulse, dynamic blood pressure, dynamic heart rate, and blood oxygenation levels. Other vital signs may be measured using this method. This invention provides for the real time transfer of data relating to vital signs to an external monitoring system. The external monitoring system is staffed by certified medical personnel. The system shall provide user location via GPS so that first responders can be notified when appropriate.

2. Description of Related Art

The invention herein relates generally to a wrist-worn monitoring device to obtain and transmit a users vital signs to an appropriate external monitoring service. Numerous devices are on the market or have been patented that disclose devices that monitor heart rate and store the date for later downloading. Existing hardware also provides the means for a user to download their own data. Nothing disclosed to date provides for the real time detection of vital signs via a wrist device and the transfer of said data in real time to an external monitoring system composed of certified health personnel. The present invention meets this medical need.

Every year there are millions of Americans that suffer from cardiovascular disease, respiratory disease, and complications therefrom. Currently, patients that suffer from these diseases are either monitored as an inpatient in a hospital setting or as an outpatient. Those monitored on an outpatient setting visit a doctor at least monthly to determine their vital signs including pulse, blood pressure, heart rate, and oxygenation levels. The vital signs obtained while in a health care setting may not accurately reflect the health condition of the patient. Typically, a patient visiting a doctor is in a sedentary position having been at rest in the waiting room prior to having their vital signs taken. The pulse, blood pressure and heart rate of a person at rest can be markedly different from the rate experienced when a person is shopping at a large department store or mopping their floor. Additionally, the oxygenation level of a patient with COPD or asthma may vary markedly depending on whether that patient is sedentary or walking their dog. There currently exists a need to better monitor patients with a medical history of cardiac disease and pulmonary disease.

Patients in a hospital are monitored continuously. But, the equipment to perform said monitoring is bulky and cumbersome. The equipment is customarily pushed around on a platform that is difficult for a patient to push through the room or through a hospital hallway. If the patient is unable to push the equipment to the bathroom, an alarm sounds requiring a nurse or other healthcare provider to attend to the patient. While the patient is being transported to another room or going to the bathroom, their vital signs are not being monitored. Incorporating these monitoring devices on a wrist device enables a patient to be monitored without the necessity for bulky equipment that is difficult or impossible for a patient to maneuver.

Although current monitoring devices obtain vital measurement data which can be stored, said devices do not communicate in real time to certified medical personnel. Current monitors disclose the ability to obtain and store relevant vital sign data and to warn a user of variations in said data. But, many patients suffering from cardiac and pulmonary disease lack the ability to perceive and understand such data. Many patients suffering cardiac and pulmonary disease are elderly and infirm. They may not appreciate when the vital signs indicated on the display of the Wrist Vital Sign Monitoring Device monitor indicate obtaining emergency medical attention. Likewise, they may not understand when they do not need to seek emergency medical attention. This device will provide its users with certified medical personnel to inform them when to obtain emergency medical attention and can even summon emergency personnel to attend to the user even if the user is unsure of their location or is unable to respond to the certified medical personnel.

Devices have been disclosed that monitor vital signs via a behind a subject's ear (Yang et al. WO 2008110788 A1) and along the head (Mathews U.S. Pat. No. 5,431,170 A). But, these devices are visibly noticeable and do not function if contact is reduced due to movement occurring during daily activities are during exercise. Users are less likely to wear a device worn about the head than they are to wear a device worn about the wrist. The wrist is an ideal location for monitoring vital signs. A wrist monitoring device is convenient, accessible, and non-intrusive. Monitors designed to be worn about the wrist are more likely to be utilized.

Ting et al. (U.S. Pat. No. 6,443,906 B1) discloses a wrist device that measures continuously a user's arterial blood pressure via a hemispherically shaped metal sensor. The shape of the sensor is such that the sensor continuously occludes one-half of the arterial artery when worn properly. The device is uncomfortable making a user unlikely to wear it on a routine basis. Additionally, the device only monitors blood pressure which limits is use. Rulkov et al. (U.S. Pat. No. 7,468,036 B1) discloses a monitoring device worn on the wrist with an optical sensor composed of a photodetector and a plurality of light emitting diodes to measure pulse rate, blood oxygenation levels, calories expended, time, distance traveled, and dynamic blood pressure. This allows just the user to receive the information and to interpret as they see fit. In fact, the user is required to program in his information, ensure proper fit, and ensure that the device is functioning properly. This device is not a viable option for many elderly and infirm patients that like the ability to program a device, properly fit the device upon his wrist, and to interpret results from said device.

Lo et al. (U.S. Pat. No. 7,547,282 B2) discloses an ultrasonic monitor worn about the wrist for measuring heart rate and pulse rate values with a readout displaying said measured values. The ultrasonic monitor of Lo et al. does not inform the user of when to seek medical treatment nor does it maintain the data. This device does not have a method to store data in a memory. Ali et al. discloses a dual-mode pulse oximeter wherein a sensor port receives a photo-plethysmographic signal as input to an internal process. A multi parameter patient monitoring system (“MPMS”) receives the oxygen saturation and pulse rate measurements through a docking station, which in turn is inserted in one or more slots. The MPMS of Ali et al. can serve as a MPMS communications interface. This device is only usable in a clinical or hospital setting. The device is not portable and could not be used by non-medically trained users. Additionally, Ali et al. discloses a handheld device that may be dropped or misplaced by a user. Klopfenstein et al. (U.S. Pat. No. 7,372,778 B2) discloses a wrist device that contains an electronic optical pulse measuring device and an electronic circuit to characterize the data generated. But Kloffenstein et al. does not disclose any means of storing or transmitting measurements to certified medical personnel.

Myllymaki (U.S. Pat. No. 5,515,858) discloses a wrist-held monitoring device that obtains the motoric activity or movement and physiological conditions such as temperature and/or electric conductivity of the sign and transmits the date via radio frequency to a centralized receiving point for alarm generation there. This device does not contemplate wireless transmission of vital signs to a centralized receiving point. Additionally, the data transmitted via Myllymaki is not the type of data relevant to medical personnel. Lee (WO 2013169014 A1) discloses a wristwatch blood pressure monitor comprising an air chamber on one side of the band that is used to ascertain blood pressure measurements. Lee discloses transmitting the measured blood pressure value through wireless communication such that the measured value can be utilized to efficiently manage a user's blood pressure. This device is unlikely to be worn on a continuous and routine basis due to it comprising a blood pressure “cuff” or sphygmomanometer device that inflates. Additionally, the person wearing the device must manually start the device on a schedule to monitor their blood pressure. Thus, if the user forgets to measure their blood pressure on the predetermined schedule, no measurements will be obtained. The present invention operates without any user input. So that irrespective of user input, the device will function properly as anticipated.

### BRIEF DESCRIPTION OF THE DRAWINGS

The figures in the drawings are briefly described as follows:

FIG. 1 illustrates the flow chart for blood pressure monitoring.

FIG. 2 illustrates the flow chart for heart rate monitoring.

FIG. 3 illustrates the flow chart for pulse rate monitoring.

FIG. 4 illustrates the flow charge for oxygen rate monitoring.

FIG. 5 illustrates the Wrist Vital Sign Monitoring Device microprocessor inputs and outputs.

FIG. 6 illustrates the calibration process for the Wrist Vital Sign Monitoring Device monitor.

FIG. 7 illustrates user data storage for the Wrist Vital Sign Monitoring Device.

FIG. 8 illustrates the transfer of data and communication system for the Wrist Vital Sign Monitoring Device monitor.

FIG. 9 illustrates sample readouts that twenty different users might see on the face of the Wrist Vital Sign Monitoring Device monitor. These sample readouts include values for both heart rate on the left of the readout and blood pressure on the right.

FIG. 10 illustrates the Wrist Vital Sign Monitoring Device monitor which can be worn about the wrist.

FIG. 11A illustrates the electrical diagram for the microprocessor showing inputs, the power supply, the transceiver that transmits data to wireless sources and computer systems.

FIG. 11B illustrates the electrical diagram for the microprocessor showing data outputs, EEPROM data storage, alarm signaling, GPS receiver, and LCD module.

FIG. 12 is a flow chart illustrating temperature sensing function.

FIG. 13 depicts the electrical circuit at the front (not in contact with the User's wrist) of the Wrist Vital Sign Monitoring Device.

FIG. 14 depicts the electrical circuit at the back (in contact with the User's wrist) of the Wrist Vital Sign Monitoring Device.

FIG. 15 depicts the electrical transistor circuit for the heart rate function.

FIG. 16 depicts the electrical transistor circuit for the temperature function.

FIG. 17 depicts sample data derived from each electrical transistor circuit in both the front and back electrical circuits (shown in FIGS. 13 and 14).

FIG. 18 depicts the calculation of the time delay between the data derived from the front electrical transistor circuit and the back electrical transistor circuit.

### DETAILED DESCRIPTION OF THE DRAWINGS

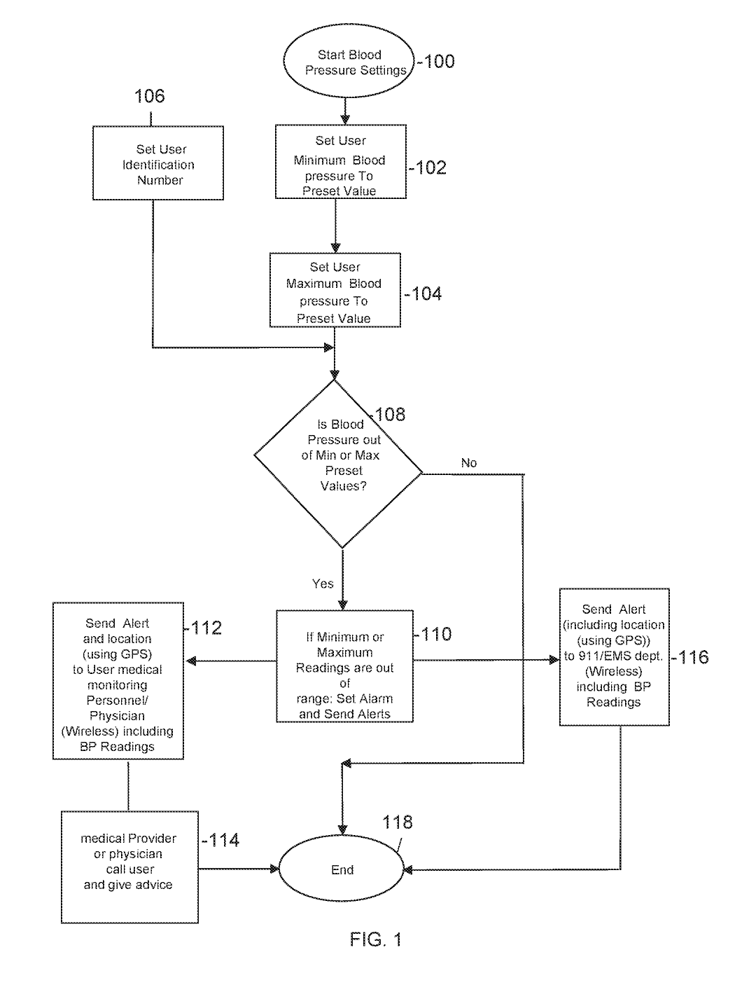

FIG. 1 depicts the flow chart for blood pressure monitoring within the Wrist Vital Sign Monitoring Device monitor. Preset values for the user's blood pressure rates will be predetermined by the user's physician or other certified health care professional and stored with the minimum value being stored at step 102 and the maximum value being stored at 104. A user identification number may be programmed into the device at step 106. Once worn by a user, the device will ascertain when a vital sign of the user is outside the range set by minimum or maximum values at step 108. If a vital sign is outside of the range set at steps 102 and 104, the device will cause the alarm to sound or send alerts as predetermined at step 110. The alert generated when predetermined minimum or maximum readings are outside of the range set by the certified health care professional may be received by certified medical personnel hired to staff the monitoring system using smart phones, 911 or other emergency medical system, or other wireless electronic media at step 116. Certified medical personnel receiving the alert will call the user and give specific medical advice as needed. Additionally, alarms and alerts may be sent to medical monitoring personnel such as a physician or a monitoring service at step 112. A physician or medical provider may contact a user and provide advice at step 114.

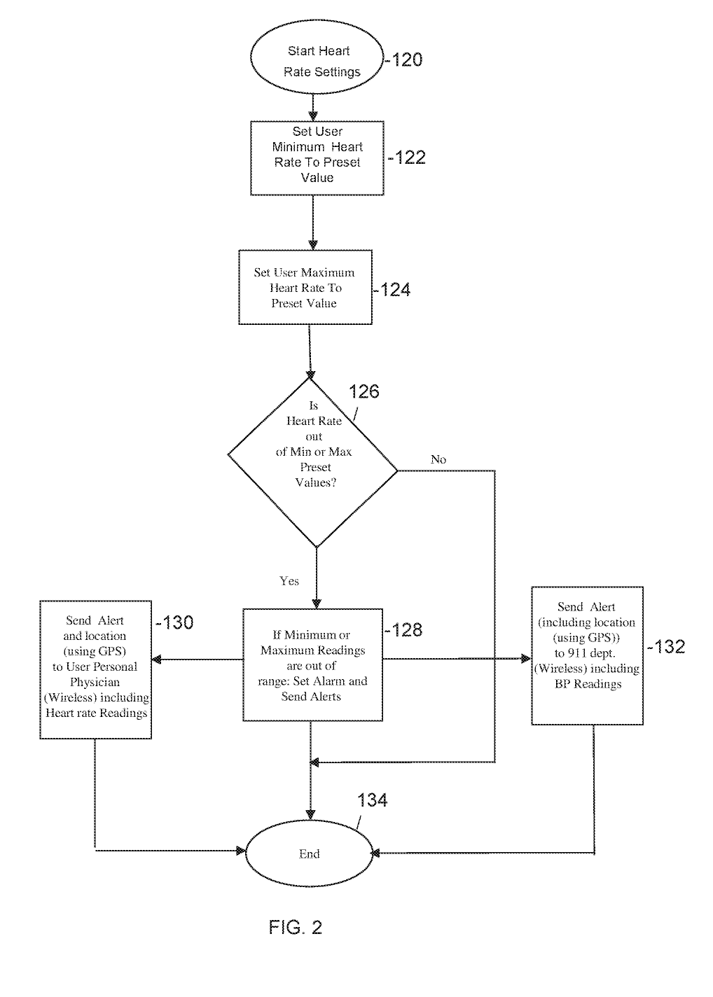

FIG. 2 illustrates the flow chart for heart rate monitoring to be performed by the Wrist Vital Sign Monitoring Device monitor. All preset values for the user's heart rate will be predetermined by the user's certified health care professional. Minimum heart rate vital signs may be preset at step 122, and maximum values preset at step 124. The device will monitor whether the user's heart rate vital sign is within the presets at steps 122 and 124. If the vital sign is outside of the preset values, then the device may produce an alarm and/or send alerts at step 128. The alert generated when minimum or maximum readings are outside of the range predetermined by the certified health care professional may be received by certified medical personnel hired to staff the monitoring system using smart phones, 911 or other emergency medical system, or other wireless electronic media at step 130. Certified medical personnel receiving the alert will call the user and give specific medical advice as needed. Additionally, the device may send an alert using GPS to local emergency personnel such as EMS or 911 at step 132.

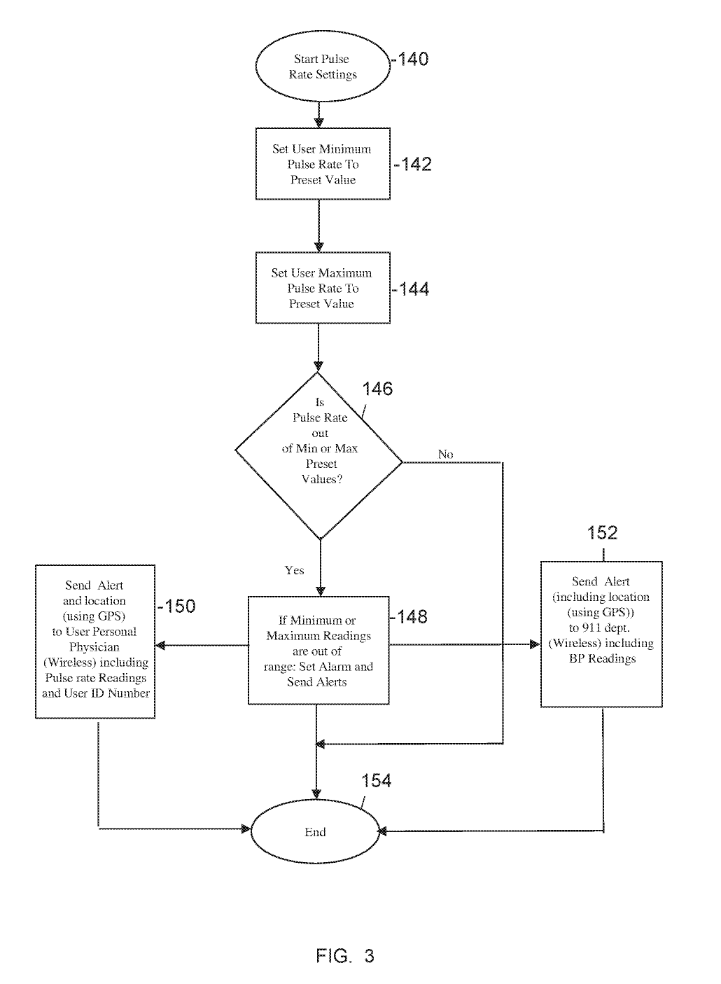

FIG. 3 illustrates the pulse rate monitoring flow chart. All preset values for the user's pulse rate will be determined by the user's certified health care professional. Minimum and maximum predetermined values for a user's pulse will be programmed into the device at steps 142 and 144, respectively. If the user's pulse is less than the minimum value or greater than the maximum value at step 146, the device may generate an alarm and/or alert at step 148. If the user's pulse is outside of the range set by the certified health care professional at step 148, the device's alarm or alert may be received by certified medical personnel hired to staff the monitoring system using smart phones at step 150, 911 or other emergency medical system, or other wireless electronic media at step 152. Certified medical personnel receiving the alert will call the user and give specific medical advice as needed.

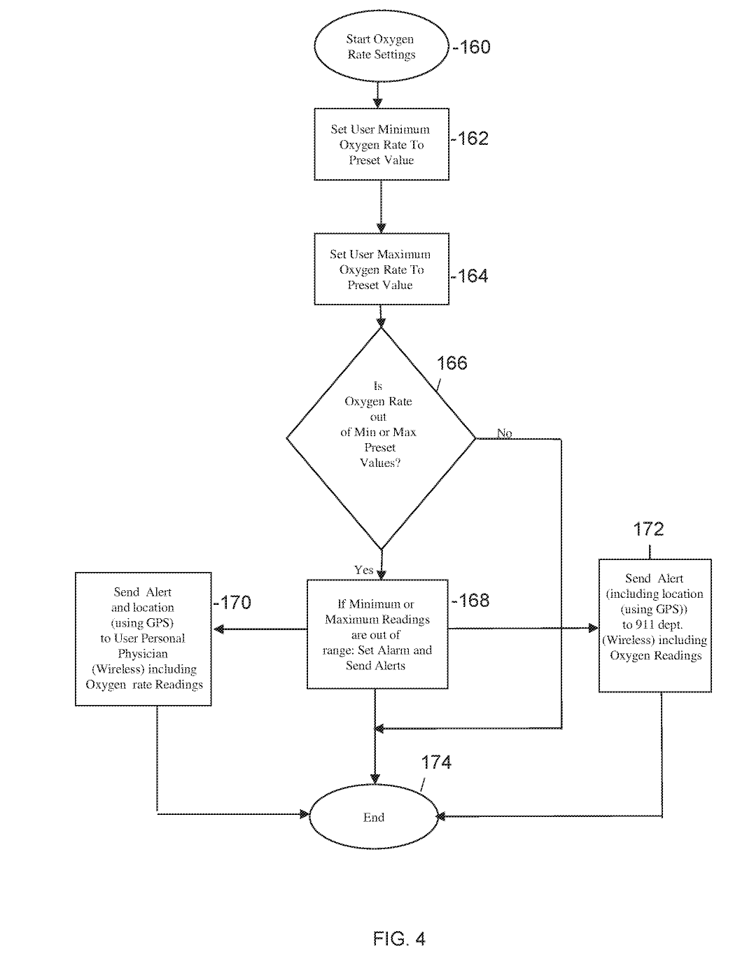

A flow chart depicting the oxygen rate monitoring function of the Wrist Vital Monitoring Device is illustrated in FIG. 4. The Wrist Vital Sign Monitoring Device monitor may contain an oximeter to determine oxygen saturation levels within the user. All preset values for the user's oxygenation levels will be determined by the user's certified health care professional and programmed into the device at steps 162 and 164. If the user's oxygen saturation is less than or more than the predetermined values at step 166, the device may generate an alarm and/or alert at step 168 to alert certified health care professionals at step 170 and may be received by certified medical personnel hired to staff the monitoring system using smart phones, 911 or other emergency medical system, or other wireless electronic media at step 172. Certified medical personnel receiving the alert will call the user and give specific medical advice as needed.

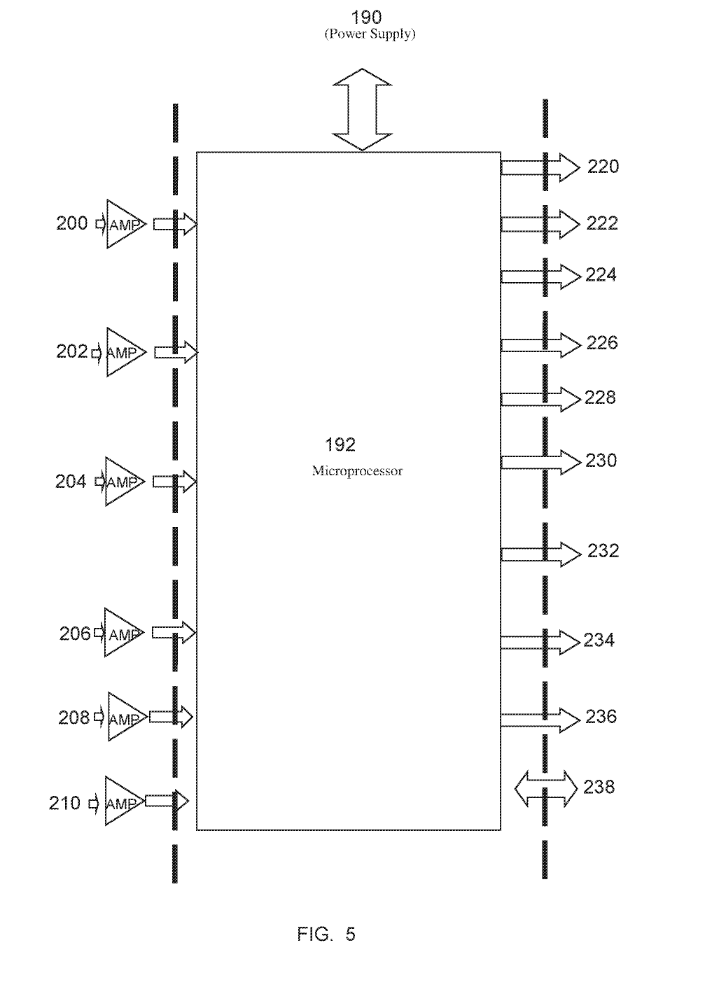

FIG. 5 illustrates the microprocessor unit utilized by the Wrist Vital Sign Monitoring Device monitor. A Motorola® 68008 microprocessor 192 would be a suitable microprocessor for use in the Wrist Vital Sign Monitoring Device monitor. Blood pressure 200, heart rate 202, pulse 204, oxygen saturation 206, body temperature 208, or other vital signs 210 may be obtained by the sensor(s) contained within the Wrist Vital Sign Monitoring Device monitor. These sensors will provide inputs to microprocessor 192. Power 190 provided to microprocessor 192 will enable microprocessor 192 to perform functions consistent with the flow charts depicted in FIGS. 1 through 4, above. Microprocessor 192 will output any necessary alert to the user, local emergency medical service providers, and the user's health care professional(s), 220, 222, 224, 226, 228, 230, 232, 234, 236, and 238. The alert to the local emergency medical service providers will include the location via global positioning satellite of the user. Microprocessor 192 will retain all data relating to acquired vital signs in volatile memory for downloading by the user or the user's health care professionals.

Microprocessor 192 will generate three levels of alert: (1) level one which alerts the patient only; (2) level two which provides an alert to both the patient and a monitoring service, and (3) level three which provides an alert to the patient, to the monitoring service, and to emergency medical personnel. GPS will inform emergency medical personnel and the monitoring service the location of the user. Alerts to the patient and to the monitoring service may be received by smart phone, reader, or other electronic device including wireless devices.

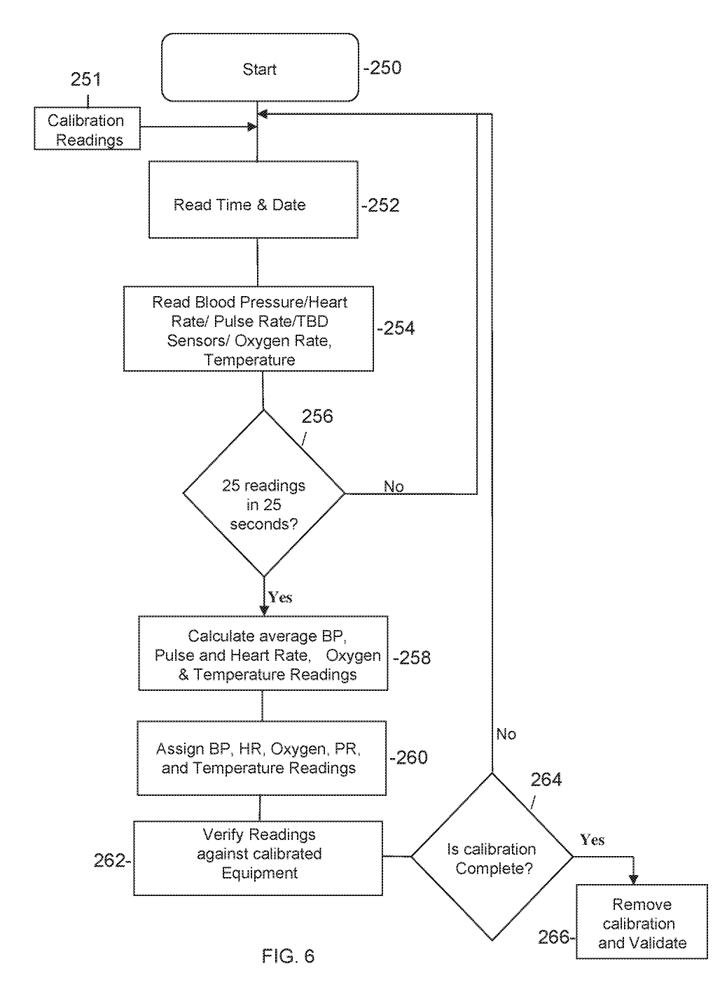

FIG. 6 illustrates the calibration process for the Wrist Vital Sign Monitoring Device monitor. The Wrist Vital Sign Monitoring Device will be calibrated so that it is able to perform at least 25 readings of vital signs within 25 seconds. Calibration readings at step 251 will allow the programming of time & date at step 252, and blood pressure, heart rate, pulse rate, oxygen rate, and other vital signs to be determined at step 254. 25 readings may be performed in 25 seconds at step 256. Upon receipt of the readings at step 256, the device will calculate the user's average blood pressure, pulse rate, heart rate, oxygen saturation, temperature and other vital signs to be determined at step 258. The device will assign blood pressure, heart rate, oxygen saturation, and pulse rate, and temperature readings at step 260. These readings will be verified at step 262. Once calibration is complete at step 264, the device will be validated at step 266.

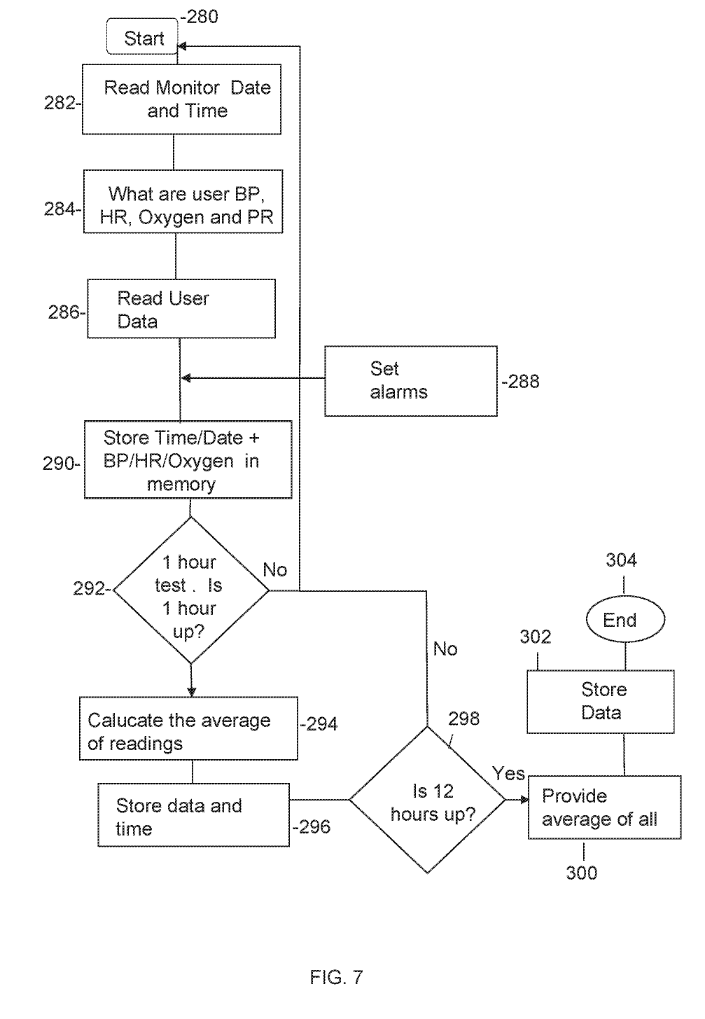

User data storage is depicted in the flow chart of FIG. 7. Alerts are to be set based on the user's health care professional's recommendations. Maximum and minimum values will be determined for at least one of the following: blood pressure rate, heart rate, pulse rate, oxygen saturation, body temperature, or other vital signs as warranted. Data storage can be performed using an Electrically Erasable Programmable Read-Only Memory (“EEPROM”) which is a type of non-volatile memory commonly used in computers and other electronic devices to store data. Calibration should be completed every six months to maintain accuracy of the Wrist Vital Sign Monitoring Device monitor.

The flow chart of FIG. 7 depicts the user data storage capacity of the Wrist Vital Sign Monitoring Device monitor. Alarms are set by certified medical personnel based on their recommended minimum and maximum values for blood pressure, heart rate, pulse rate, oxygenation state, body temperature, etc. The device will be set so that it reads the date and time at step 282, the vital signs taken at step 284, the user's data is read at step 286, and stored at step 290. The testing performed at steps 282, 284, and 286, may be performed for one hour at step 292 to calculate an average reading of data per user at step 294. The user's average reading is stored in EEPROM at step 296. Data and the time of each reading is maintained and an average of the data is computed and transmitted every twelve hours at step 298. The average of all data is calculated at step 300 and stored at step 302.

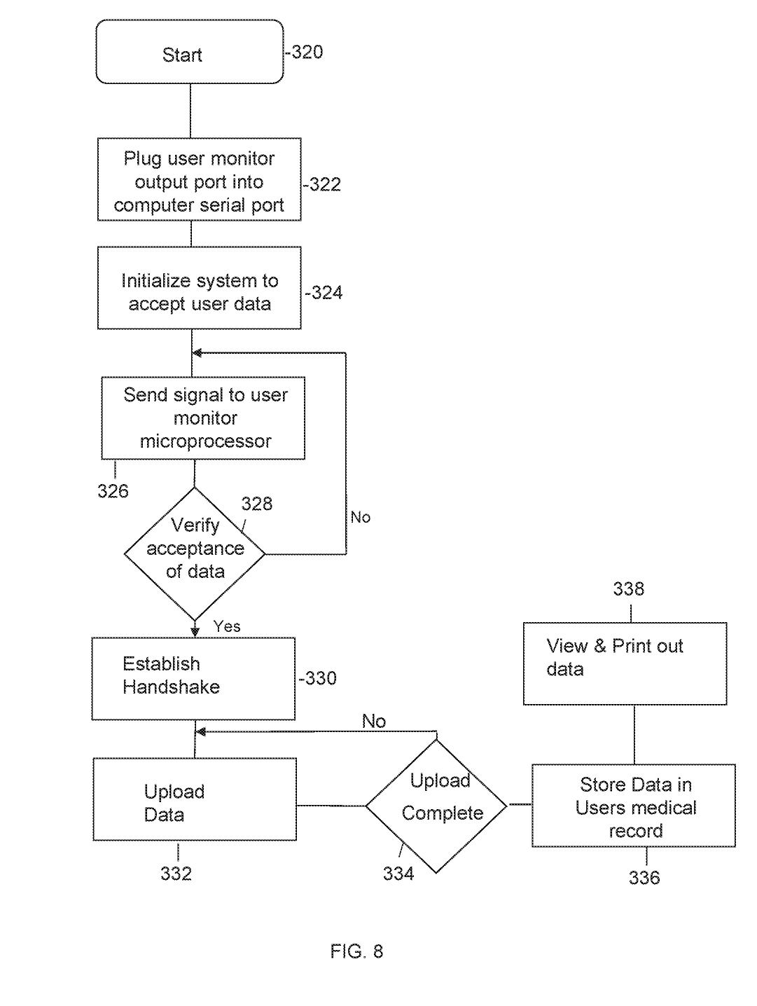

Data transfer and communication is depicted in FIG. 8. The output port of a user's monitor may be plugged into a computer serial port at step 322. At step 324, the system will be initialized to accept the user's data. A signal will be sent to the microprocessor of the user's monitor at step 326. Next the acceptance of data will be verified at step 328 to establish a handshake at step 330. Once the handshake is established at step 330, the user's data may be uploaded at step 332. The vital sign data may be uploaded and stored in a medical records database in EEPROM at step 336 so that it can be viewed by the user's certified medical provider(s) at step 338. This allows the user's physician to determine the cause of changes in the user's environment that may have contributed to alerts being generated.

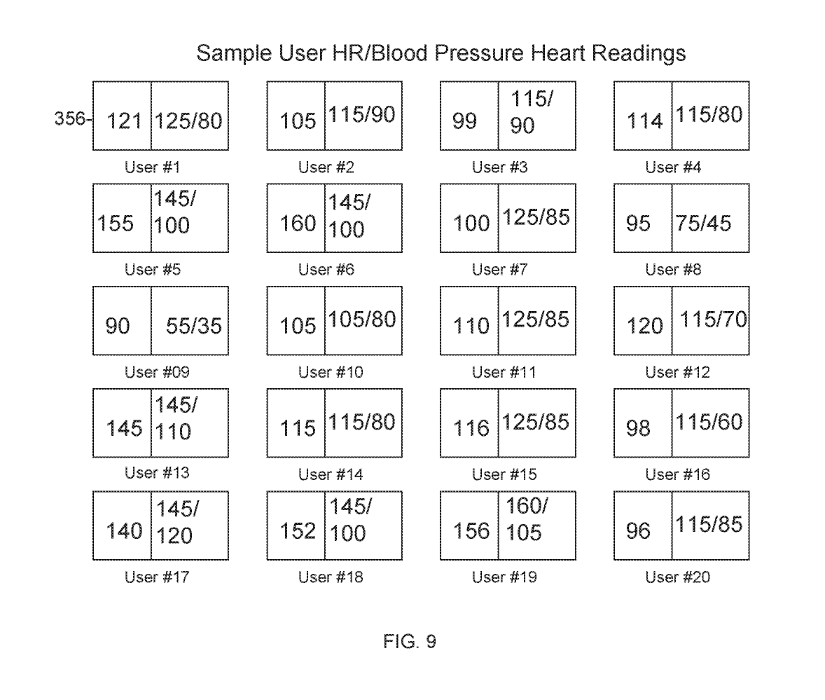

FIG. 9 depicts sample vital signs 356 for hypothetical users numbered one through twenty (User #1-User #20). The device may display heart rate and blood pressure for each user. The left half of each box shown in FIG. 9 displays the heart rate and the right half of each box displays the blood pressure reading for each user.

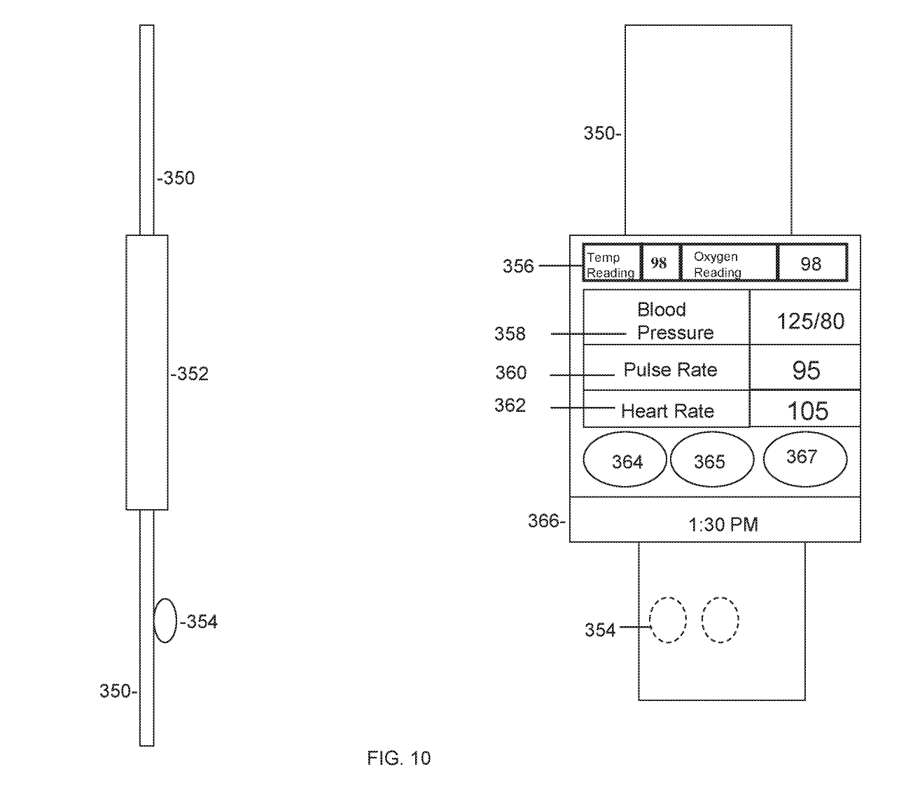

The Wrist Vital Sign Monitoring Device monitor is depicted in FIG. 10. The Wrist Vital Sign Monitoring Device monitor is designed to be worn about the user's wrist. It is designed to be lightweight, thin, and easy to calibrate and read. The Wrist Vital Sign Monitoring Device is composed of two straps 350 that connect the monitor display 352. The bottom strap 350 attaches one or more sensors 354 to detect heart rate 362, blood pressure rate 358, pulse rate 360, temperature 364, time 366, and in some embodiments, oxygenation saturation levels. Strap 350 is a means for securely holding the Wrist Vital Sign Monitoring Device's sensor(s) in contact with the user's body at a location adjacent to a selected artery. The strap is designed to curve so that the strap will comfortably maintain contact with the user's body.

Sensors 354 may be optical electronic sensors, metal sensors, or other sensors adapted to continuously monitor a user's vital signs and to generate signals representative thereof by contact with an external surface of the user's body that is adjacent to the user's artery. The sensor is to be positioned at the optimal position on the back of the Wrist Vital Sign Monitoring Device by a certified medical professional so that the optimal location can be acquired. Strap 350 may be modified to fit to the user. For example, spacers may be added to strap 350 to provide for a better fit for the user. Flexion and extension of the wrist can cause the sensor(s) 354 to move from their optimal location. Therefore, it is utmost importance to instruct the user of the position that must be maintained for the Wrist Vital Sign Monitoring Device to properly function. Excessive tightness of strap 350 can cause edema which may affect the sensor's ability to accurately obtain signals. Thus, the device should be fitted properly and reassessed frequently for fit especially upon any weight gain or loss of the user.

The Wrist Vital Sign Monitoring Device may include wireless antenna 365 to enable the wireless transmission of data and alerts, speaker 367 for audio discussions between the user and the monitoring service and the user's certified medical professional, an audio alarm to alert the user when vital signs are detected that are outside the predetermined vital signs ranges set by certified medical professionals, a functionality button to program the Wrist Vital Sign Monitoring Device and to reset functions, and a display face to display dynamic readings of certain vital signs. In one embodiment, the functionality button can only be programmed or reset by the monitoring system or by certified medical professionals. The face of the device can display dynamic and average heart rate, blood pressure rate, pulse rate, and oxygen saturation level. Additional vital signs such as body temperature can be displaced on the face of the Wrist Vital Sign Monitoring Device. Time is displayed and vital signs acquired are associated with the time set on the display face. The time function is integrated with the other vital sign functions so that data relating to vital signs can be correlated to time, which allows a user to correlate the time and date with what activity the user was participating in. This informs the user of what types of activities he or she should or should not participate in.

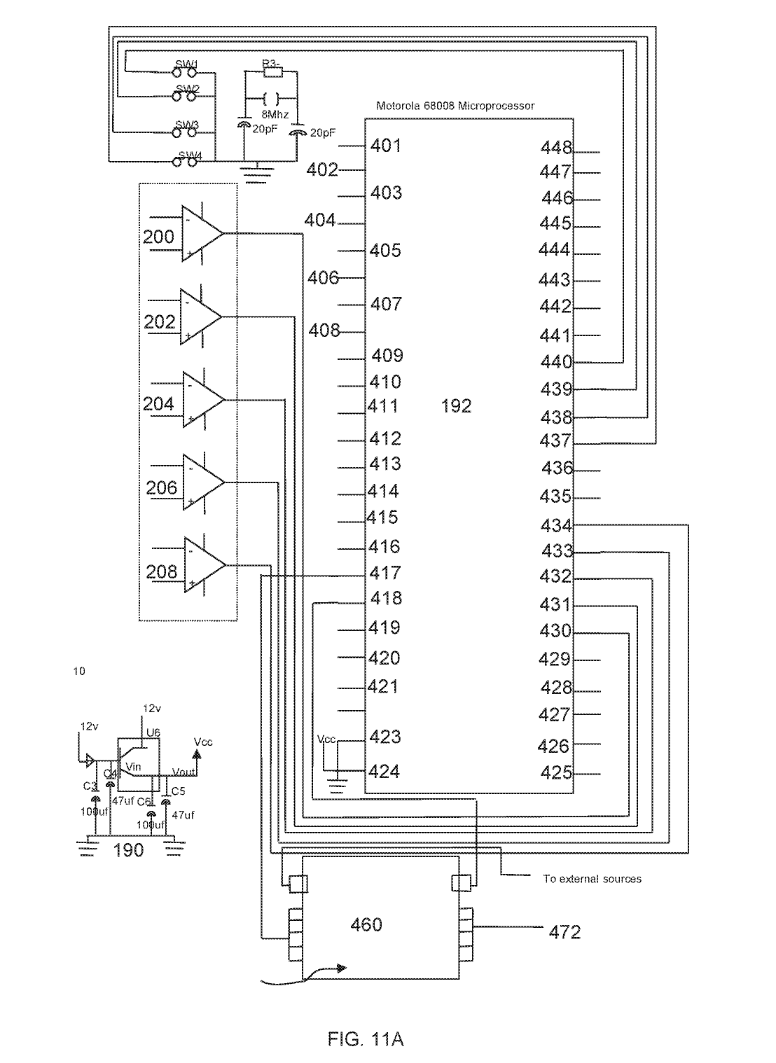

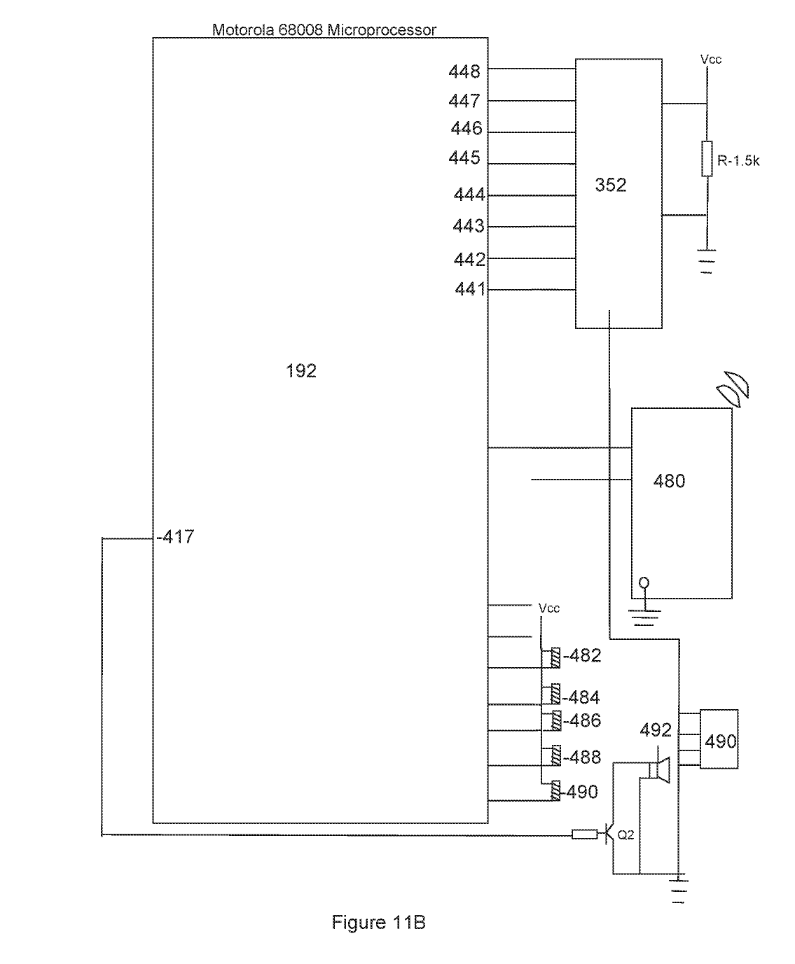

The microprocessor is contained within the watch beneath monitor display 352. Microprocessor 192 contains the means for interpreting signals from the sensor depicted in FIG. 10. The electrical diagram for microprocessor 192 is depicted in FIGS. 11A and 11B. Microprocessor 192 interprets signals received from sensor(s) 354 and computes dynamic rates for the preprogrammed vital signs being measured. FIG. 11A depicts five inputs obtained from sensor(s) 354, these sensors may include: blood pressure 200, heart rate 202, pulse 204, oxygen saturation 206, body temperature 208, or other vital signs as desired. More inputs may be programmed into the Wrist Vital Sign Monitoring Device. These sensor inputs transmit information to microprocessor 192, which translates the input into data at 401, 403, 405, 407, 409, 410, 411, 412, 413, 414, 415, 416, 417, 418, 419, 420, 421, 423, 424, 425, 426, 427, 428, 429, 430, 431, 432, 433, 434, 435, 436, 437, 438, 439, 440, 441, 442, 443, 444, 445, 446, 447, and 448. The transceiver 460 may transfer vital sign data to other devices 472 including, but not limited to, wireless devices, computers, and the external monitoring service. The electrical diagram for power supply 190 that provides power to microprocessor 192 is shown in FIG. 11A.

FIG. 11B depicts the electrical diagram showing output from microprocessor 192. Upon processing of data received from the sensor(s) 354, data is transmitted out of microprocessor 192 via 441, 442, 443, 444, 445, 446, 447, and/or 448 to the LCD or LED module 352 on the front of the Wrist Vital Sign Monitoring Device. Additionally, data may be transmitted to GPS receiver 480. Data obtained is stored in EEPROM data storage and maintained for future downloading or transmission. When vital signs are obtained outside of the predetermined range, alarms within the monitor 417 may be generated by microprocessor 192 to alert the user, the monitoring service, and emergency personnel if need. These alarms may include a temperature alarm 482, blood pressure alarm 484, heart rate alarm 486, pulse rate alarm 488, and/or temperature alarm 490. Buzzer 492 may be included with the device to alert the user of a vital sign. The occurrence of an alarm(s) and/or buzzer 492 may be stored in EEPROM data storage 490. The Wrist Vital Sign Monitoring Device may be powered by battery including, but not limited to, solar power, lithium batteries, or any other suitable means of power.

The receiver interface allows sports teams to monitor each player's vital signs. This could be of great use for a team with one or more players suffering from asthma, which causes numerous deaths each year in the United States. Parents can also use their smart phones to monitor their child's condition per vital signs using their smart phone, electronic reader, or personal computer.

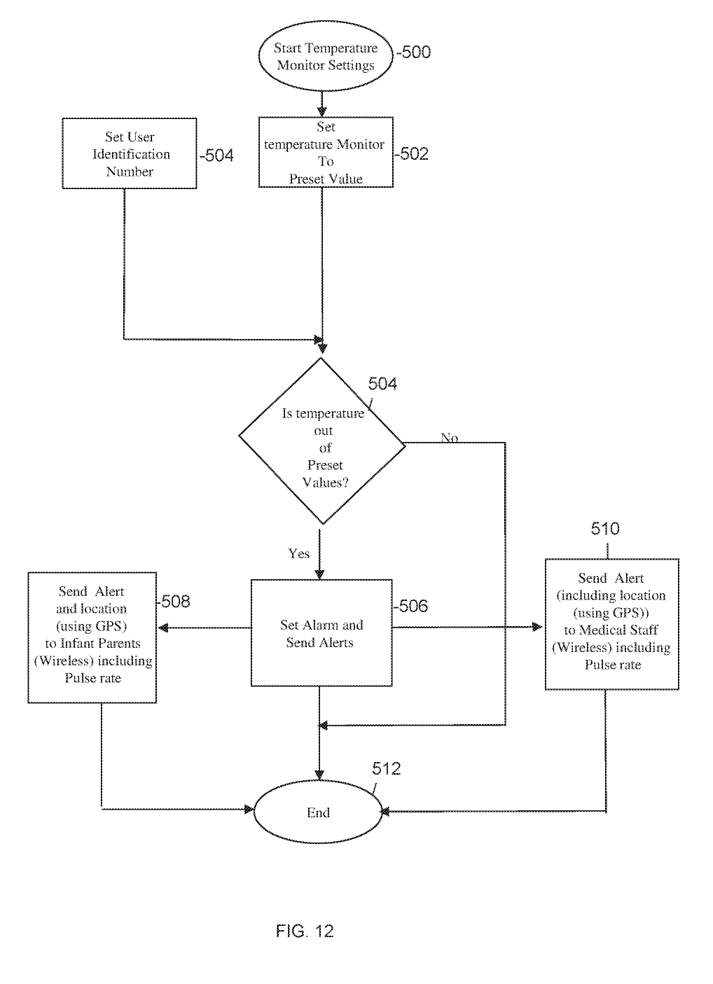

FIG. 12 is a flow chart illustrating the temperature sensing function. The minimum and maximum temperature values are preset at step 502. The user's identification number may also be set at step 504. If the temperature of the user's falls outside of the preset value at step 504, an alarm sounds and alerts may be sent at step 506. Alarms may be sent to the nurse's station, a sport's trainer, a parent, and/or a physician at step 508, or to emergency personnel at step 510. At steps 508 and 510 GPS may inform emergency personnel of the location of a user when an alert is transmitted. Temperature sensing is used in combination with the other sensors of the device to ascertain whether the user is experiencing a health crisis and is used to calculate the daily temperature values for a user.

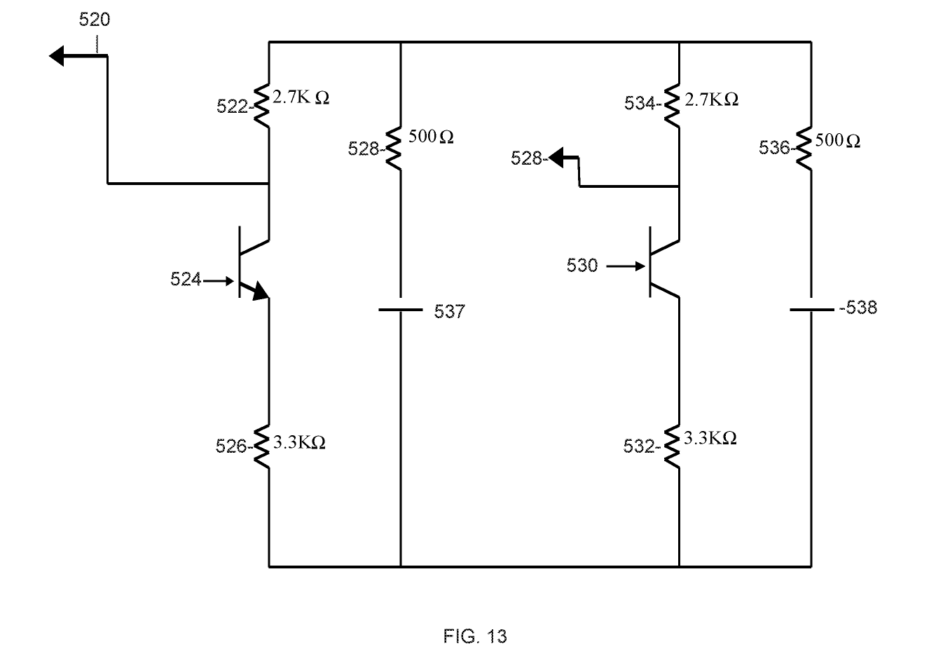

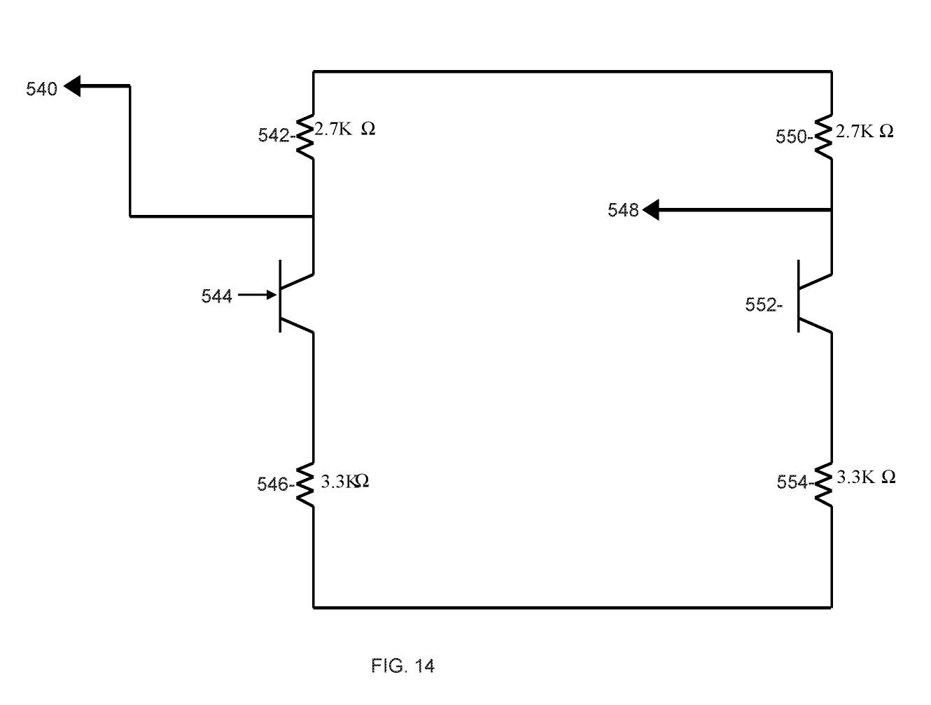

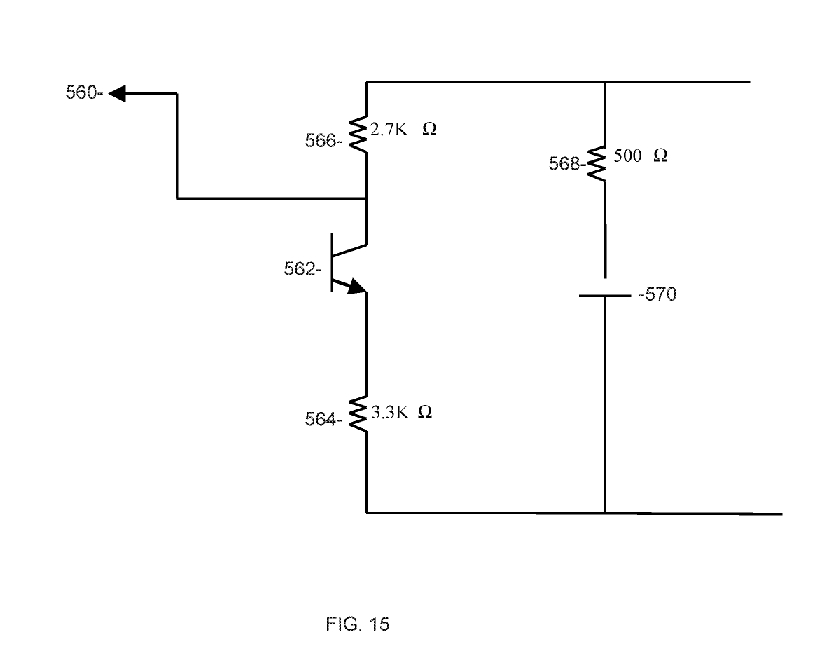

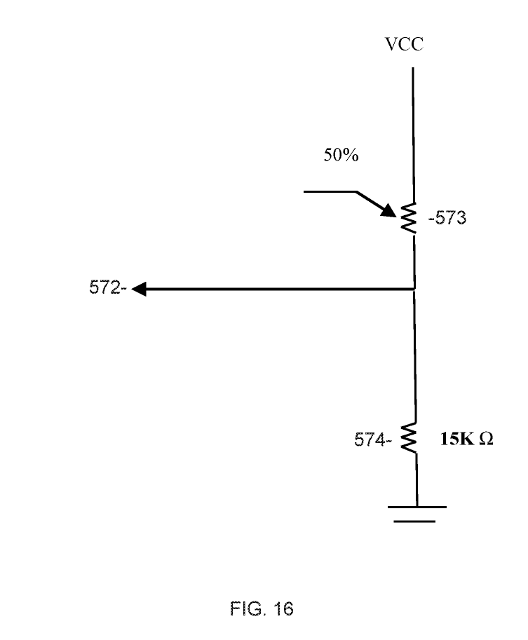

The Wrist Vital Monitor Device contains two electrical circuits-one at the front of the device and one at the back of the device. The front of the device includes monitor 352, while the back of the device is positioned against the skin of the User. The Wrist Vital Monitor Device may include 2 light emitting diodes (LEDs) 537 and 538 (FIG. 13), 15 resistors which may include: 522, 526, 528, 534, 532, 536 are shown on FIG. 13, 542, 546, 550, and 554 are seen on FIG. 14, 564, 566, 568 shown on FIG. 15; 574 shown on FIG. 16; and 4 phototransistors: red phototransistor 524, blue phototransistor 530 which are shown in FIG. 13, and red phototransistor 544 and blue phototransistor 552 which are shown in FIG. 14. The LEDs, resistors, and phototransistors are arranged in two electrical circuits. FIG. 13 depicts the electrical circuit at the top of the device (not in contact with the user's skin), and FIG. depicts the electrical circuit at the bottom of the device which is in contact with the user's skin Each electrical circuit includes a red phototransistor and a blue phototransistor to shine different spectra of light. The use of two different colors of phototransistors prevent as much interference as possible. One LED in each circuit is paired with one phototransistor to produce a blood pressure reading. For example, in FIG. 13, LED 537 is paired with red phototransistor 524 to compute the diastolic blood pressure and LED 538 is paired with blue phototransistor 530 to calculate systolic blood pressure. The user's pulse is measured for each LED/phototransistor pairing in FIG. 13. Both LEDs 537 and 537 include a transistor/circuit in both the top electrical circuit shown in FIG. 13 and the bottom electrical circuit shown in FIG. 14. Analog-to-digital circuit 520 provides the circuit for red phototransistor 524 and LED 537, while analog-to-digital circuit 528 provides the circuit for blue phototransistor 530 and LED 538. Each transistor also measures a user's pulse.

FIG. 15 illustrates the electrical transistor circuit for the heart rate function. The device utilizes red phototransistor 524 and LED 537 and sensor 354 to calculate blood pressure. The electrical circuit for red phototransistor 524 and LED 537 is shown in FIG. 15. FIG. 16 illustrates the electrical transistor circuit for the temperature function of the Wrist Vital Sign Monitoring Device. The body temperature circuit may be a voltage divider circuit, with thermistor 573. The body temperature function includes resistor 574 and is on analog-to-digital circuit 572. The change in resistance allows the temperature to be determined.

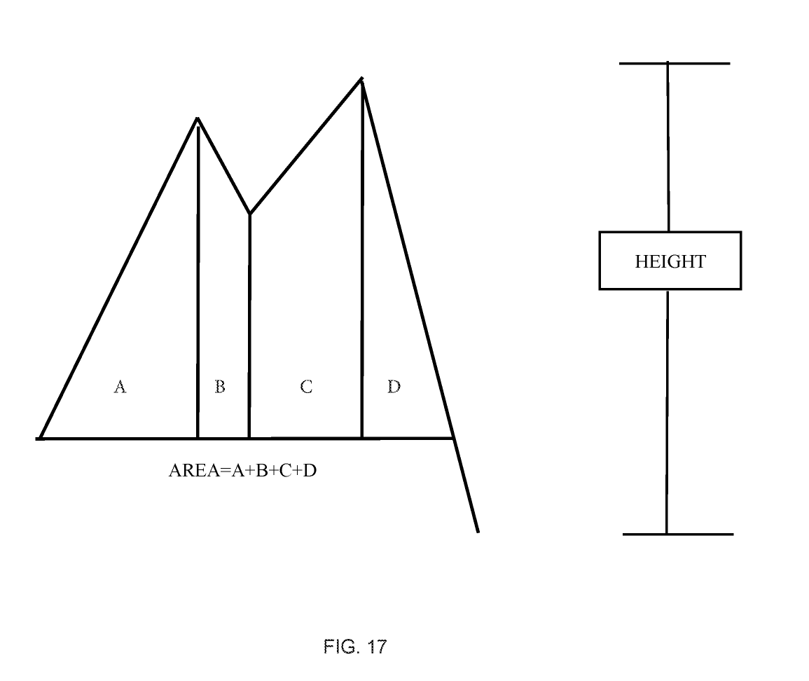

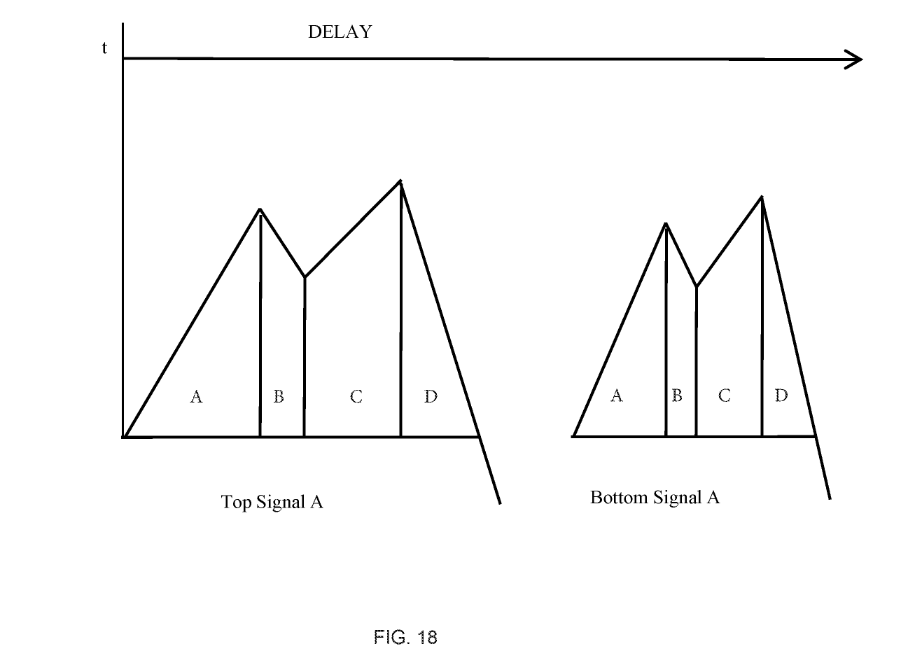

FIGS. 17 and 18 depict the method of calculating a user's blood pressure. Both the red phototransistor 524 and LED 537 and blue phototransistor 530 and LED 538 pairs (hereinafter “red and blue circuit pairs”) calculate the are of each triangle A, B, C, and D. The area of the two triangles is determined by adding A+B+C+D. The area is calculated by placing the signal received against a known template. The height of the triangle from its lowest to highest point is calculated as shown in the graph. Both the red and blue circuit pairs measure the same amount of data. The top circuit phototransistors 524 and 530 will have a different reading than top circuit phototransistors 524 and 530. The signal from top circuit phototransistors 524 and 530 will be slightly delayed and the peak will be smaller. This allows for the measurement of time between the top circuit phototransistors 524 and 530 and the bottom circuit phototransistors 524 and 530. The average of five pulses is the amount of data necessary to obtain a single blood pressure reading. The average of each of the three data points is calculated for each of the red and blue circuit pairs. The data points obtained include: the average time delay, the average height of the blood pulse, and the average area of the blood pressure. The three values obtained from each of these data points are placed in an algorithm to calculate the blood pressure of the user. The algorithm requires calibration from two separate blood pressure measurements because of differences in an individual's physiology. The algorithm is adjusted precisely for each user.

We hereby claim:

1. A monitor for measuring blood pressure values in a living subject comprising:
a power source;
one or more sensors to detect the living subject's blood pressure,
wherein each sensor is connected to two separate electrical circuits, and each electrical circuit comprises a phototransistor a light emitting diode, and one or more resistors, wherein the two separate electrical circuits are comprised of one electrical circuit that includes a red phototransistor and one electrical circuit that includes a blue phototransistor;
a microcontroller responsive to said sensor and electrical circuits, which detects and calculates the blood pressure rate;
a display unit, responsive to the microcontroller, which displays a readout of the blood pressure rate; and
a storage means to store calculated blood pressure rates.

2. The monitor of claim 1 that is designed to be worn about the wrist.

3. The monitor of claim 1 that also includes a thermistor to detect the temperature of a living subject.

4. The monitor of claim 1 that also detects the pulse of a living subject.

5. The monitor of claim 1 that also detects the heart rate of a living subject.

6. The monitor of claim 1 that includes a GPS function that allows the location of the monitor to be detected.

7. The monitor of claim 1, wherein the monitor produces an alarm or alert to notify the user if the vital sign being calculated is outside of a predetermined range.

8. The monitor of claim 1, wherein the monitor produces an alarm or alert to notify medical personnel if the vital sign of a user being calculated is outside of a predetermined range.

9. The monitor of claim 1, wherein the monitor produces an alarm or alert to notify emergency personnel, such as police or emergency medical services, that the vital sign of a user is outside of a predetermined range.

10. The monitor of claim 1, wherein the electrical circuit that includes the red phototransistor is positioned within the monitor above the electrical circuit that includes the blue phototransistor.

11. The monitor of claim 10, wherein the microcontroller calculates blood pressure by comparing the height of the signal received from the red phototransistor to the height of the signal received from the blue phototransistor.

12. The monitor of claim 10, wherein the microcontroller calculates blood pressure by comparing the area beneath the signal received from the red phototransistor to the area beneath the signal received from the blue phototransistor.

13. The monitor of claim 10, wherein the microcontroller calculates blood pressure by comparing the time delay between the signal received from the red photoresistor and the signal received from the blue phototransistor.

14. The monitor of claim 3, wherein the microcontroller calculates body temperature by comparing the change in the resistance of the thermistor.

15. The monitor of claim 5, wherein the microprocessor calculates heart rate by comparing data obtained from the red phototransistor.

16. A method of monitoring a living subjects vital signs comprising:
a monitoring device worn about the wrist that calculates vital signs;
said monitoring device continuously monitors the vital signs of a living subject 24 hours a day;
the vital signs obtained are associated with the day and time each said vital sign was obtained;
storage of said vital signs obtained for later download into a medical file; and
the transmission of wireless alarms or alerts to a physician or other medical staff if a vital sign of a living subject falls outside of a predetermined range.
